(19) United States
(12) Patent Application Publication
(10) Pub. No.: US 2004/0102906 A1
(43) Pub. Date: May 27, 2004

(54) Image processing of mass spectrometry data for using at multiple resolutions

(75) Inventor: Heinrich Roder, Steamboat Springs, CO (US)

Correspondence Address: Gary B. Solomon, Esq., Patton Boggs, LLP, Suite 3000, 2001 Ross Avenue, Dallas, TX, 75201, US

(73) Assignee: Efeckta Technologies Corporation

(21) Appl. No.: 10/646,072

(22) Filed: Aug. 22, 2003

Related U.S. Application Data
(60) Provisional application No. 60/405,399, filed on Aug. 23, 2002.

(51) Int. Cl. G06F 19/00

(52) U.S. Cl. 702/22

(57) ABSTRACT

A system and method for utilizing an image processing technique to transform raw data collected by a mass spectrometer into a hierarchical data format. The image processing technique may include the use of a wavelet transform. The hierarchical data format, provides for using the transformed data at multiple resolutions without data loss for such operations as data mining, matching, and displaying, for example. Further, the transformed data enables higher levels of data compression than generally possible from directly compressing the raw data. Additionally, the transformed data provides can be used to identify and suppress noise.

### CROSS REFERENCE TO PREVIOUS APPLICATION

This application is related to, and claims the benefit of the filing date from, U.S. Provisional Patent Application Serial No. 60/405,399, filed Aug. 23, 2002, which is herein incorporated by reference.

### BACKGROUND OF THE INVENTION

1. Technical Field of the Invention

The principles of the present invention relate to mass spectrometry, and more particularly, but not by way of limitation, to performing an image processing transform on raw data collected by a mass spectrometer.

2. Description of Related Art

Modern mass spectrometry has developed greatly in terms of the breadth of industries and technologies that use mass spectrometers to identify compounds. Examples of uses of mass spectrometers include identifying chemical and biomaterial compounds, such as DNA and blood samples. Processing the data collected by mass spectrometers has been difficult due to the volume of data collected during any given mass spectrometer run. For Example, a single mass spectrometer run typically captures 10,000 data points (having as much as one gigabyte per second of data capture rates). In the case of time-of-flight mass spectrometers, each data point includes an arrival time (proportional to the square root of mass/charge ratio) and a count of this arrival time, thereby yielding a total number of fragments having specific mass charge ratios.

There are several limitations and problems arising from the high volume of raw data collected by mass spectrometers, including time-of-flight mass spectrometers. First, viewing only the peak data signal 102 limits the ability to identify various features in the data. For example, a chemical contaminant may appear to be a trace element measured by the mass spectrometer. Also, because of the large range of scale of the vertical axis generally necessary to display the peak data signal 102, smaller measured trace elements maybe difficult to distinguish from noise. Second, most mass spectrometers are incapable of storing the large volume of raw data for later recovery or post processing investigation of the data. Third, even if a mass spectrometer includes a large enough storage unit, handling and manipulating the large amount of stored raw data is excessively time consuming. Moreover, the raw data typically proves to be difficult to use in distinguishing certain features. Fourth, using the large amount of raw data for operations or applications, such as data mining, searching, and matching, for example, is time consuming to the point of being cost prohibitive. Fifth, conventional data compression techniques, such as WINZIP, generally are complicated and do not afford benefits beyond data compression of datasets in their entirety, thereby limiting the amount of data compression possible. Also, because FDA regulations are now requiring the complete raw data to be made available at later dates, lossless compression and higher levels of data compression than possible with conventional data compression techniques are needed.

### SUMMARY OF THE INVENTION

To overcome the problems and limitations of conventional mass spectrometers for collecting and processing raw data, the principles of the present invention utilize an image processing technique for transforming the raw data into a hierarchical data format. The image processing technique may include the use of a wavelet transform. The hierarchical data format of the transformed data allows the transformed data to be used at multiple resolutions without data loss for such operations as data mining, matching, and displaying, for example. Further, the hierarchical data format of the transformed data enables higher levels of data compression than generally possible from directly compressing the raw data. Additionally, the hierarchical data format of the transformed data provides for identifying and suppressing noise generally better than possible directly from the raw data.

In a further embodiment, the principles of the present invention provide for a mass spectrometer system having a data acquisition unit operable to sense and generate raw data indicative of masses of particles. The mass spectrometer system further includes a computing unit configured to receive and transform the raw data into transformed data having a hierarchical data format for use at multiple resolutions. In one embodiment, the transformation includes the use of a wavelet transform as understood in the art. In another embodiment, the wavelet transform may use a data-adaptive technique to optimize filters utilized for the wavelet transformation over local regions.

The processing unit may be further configured to decode the transformed data at a selectable resolution for a variety of uses, such as displaying, searching, and matching, for example, to offer research or data mining capabilities that are difficult or substantially impossible to achieve by using the raw or peak data.

### BRIEF DESCRIPTION OF THE DRAWINGS

The principles of the present invention will be described with reference to the accompanying drawings, which show important sample embodiments of the invention and which are incorporated in the specification hereof by reference, wherein:

FIG. 1 is a graph of an exemplary peak data signal produced by a single time-of-flight mass spectrometer run;

FIG. 2 displays a collection of raw data of a time-of-flight mass spectrometer that is collected while the input of the mass spectrometer is fed by a front end separation engine;

FIG. 3 is a block diagram of an exemplary time-of-flight mass spectrometer that may be used in accordance with the principles of the present invention;

FIGS. 4-7 are graphs of increasing coarsened levels (i.e., multiple resolutions) of the raw data of FIG. 2;

FIG. 8 is a graph of the exemplary raw data of FIG. 2 after denoising;

FIG. 9 is a graph of an exemplary peak data signal, including raw data, denoised data, and noise data, produced by the time-of-flight mass spectrometer of FIG. 3;

FIG. 10 is a flow diagram of an exemplary process for applying a wavelet transform to the raw data of the mass spectrometer of FIG. 3;

FIG. 11 is a block diagram of exemplary software modules utilizing the processing of FIG. 10;

FIG. 12 is a flow diagram of an exemplary process for producing the transformed data having the hierarchical data format utilizing the software of FIG. 11;

FIG. 13 is a graph showing an exemplary data signal for use in interpolating a data point using the software of FIG. 11;

FIG. 14 illustrates production of the transformed data having the hierarchical data format utilizing a data-adaptive wavelet transform as may be performed by the software of FIG. 11;

FIG. 15 illustrates an exemplary decoder utilized to receive the output of FIG. 14 to reproduce the transformed data produced by the data-adaptive wavelet transformation of FIG. 14;

FIG. 16 is a flow chart describing an exemplary method for generating the transformed data having a hierarchical data format by utilizing a data-adaptive wavelet transform as illustrated in FIG. 14;

FIG. 17 is a block diagram of an exemplary configuration of the mass spectrometer in communication with an external computer system; and

FIG. 18 is a flow diagram of an exemplary procedure for using the transformed data in the hierarchical data format collected by the mass spectrometer of FIG. 17 for a variety of operations.

### DETAILED DESCRIPTION OF THE EXEMPLARY EMBODIMENTS

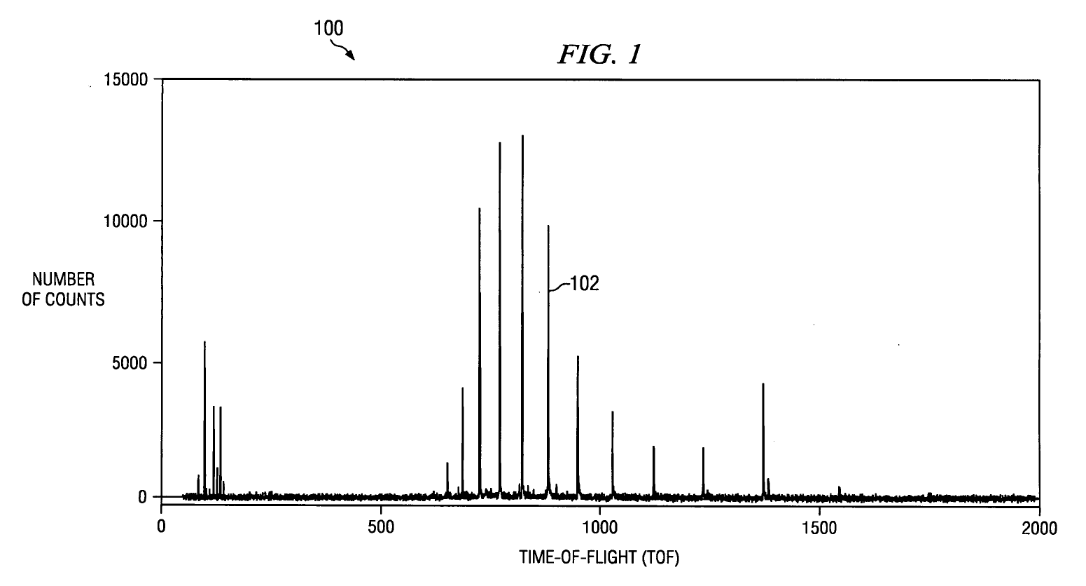

FIG. 1 is a graph or plot 100 of an exemplary peak data signal produced by a single time-of-flight mass spectrometer run. As shown, the plot 100 displays a peak data signal 102 representative of the sensed particles captured by the mass spectrometer. The peak data signal 102 is displayed as the number of counts versus time-of-flight. The time of flight of the sensed particles measures the M/Z ratio. The peak data signal 102 includes several peaks 104 that indicate that a certain number of particles (e.g., 12,500) took a certain amount of time to travel from an initiation point to a sensor of the mass spectrometer. The peak data signal 102 is formed essentially of the peak total counts produced by the cumulative sampling of ionized particles. As understood in the art, peak data signals 102 are based on a raw dataset as shown in FIG. 2 and are typically utilized because collecting and storing the total volume of raw data is generally prohibitive in terms of processing bandwidth and storage capacity limitations.

FIG. 2 displays a collection of raw data of a time-of-flight mass spectrometer that is collected while the input of the mass spectrometer is fed by a front end separation engine, in this case liquid chromatography. The horizontal axis corresponds to the time-of-flight coordinate and the vertical axis corresponds to the number of the mass spectrometer run being synchronized with the front end. As understood in the art, the individual peaks 104 of FIG. 1 are produced by correlating darker spectral lines 202 extending vertically, which is related to the elution time of the front end apparatus. Similar pictures are also obtained when a single sample is run many times to improve the statistics of the data collection engine of the mass spectrometer. The lighter spectral lines 204 represent samples at certain times-of-flight, but fewer than the number of samples collected at the times that form the darker spectral lines 202. Dark spots 206 may be indicative of chemical contaminants, systematic noise, and/or other measurement artifacts. However, the dark spots 206 are often difficult to see in the vast amount of raw data produced by the mass spectrometer. Other visual aberrations, such as underlying Moire patterns (not shown) may be due to voltage/interleaving fluctuations arising from the A/D conversion process in the data acquisition system of the time-of-flight spectrometer.

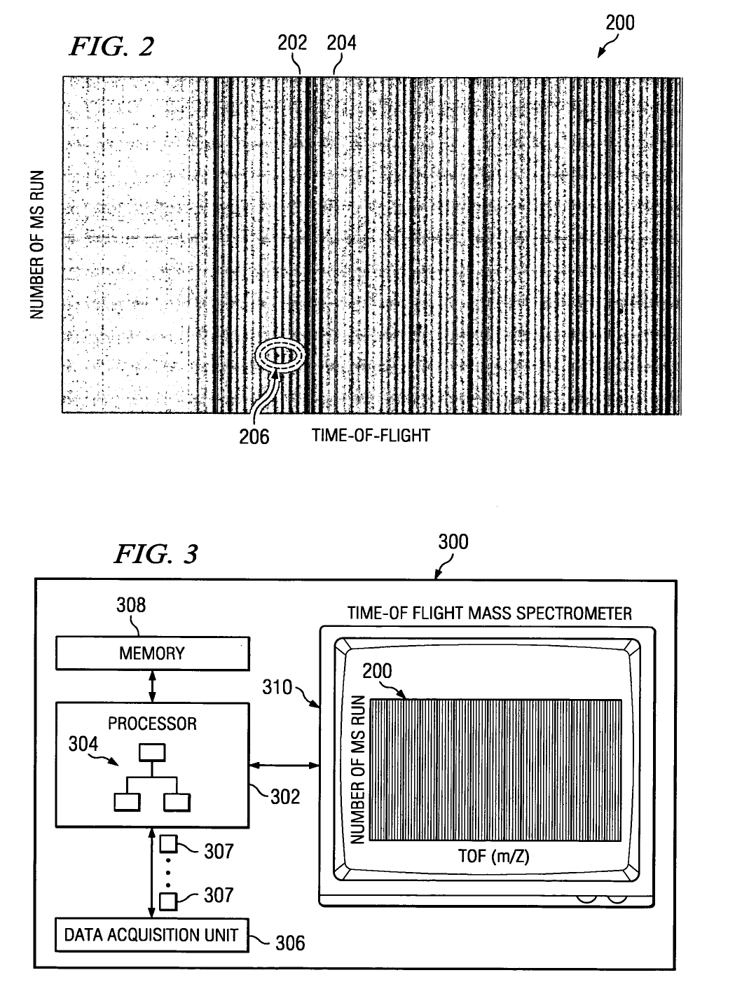

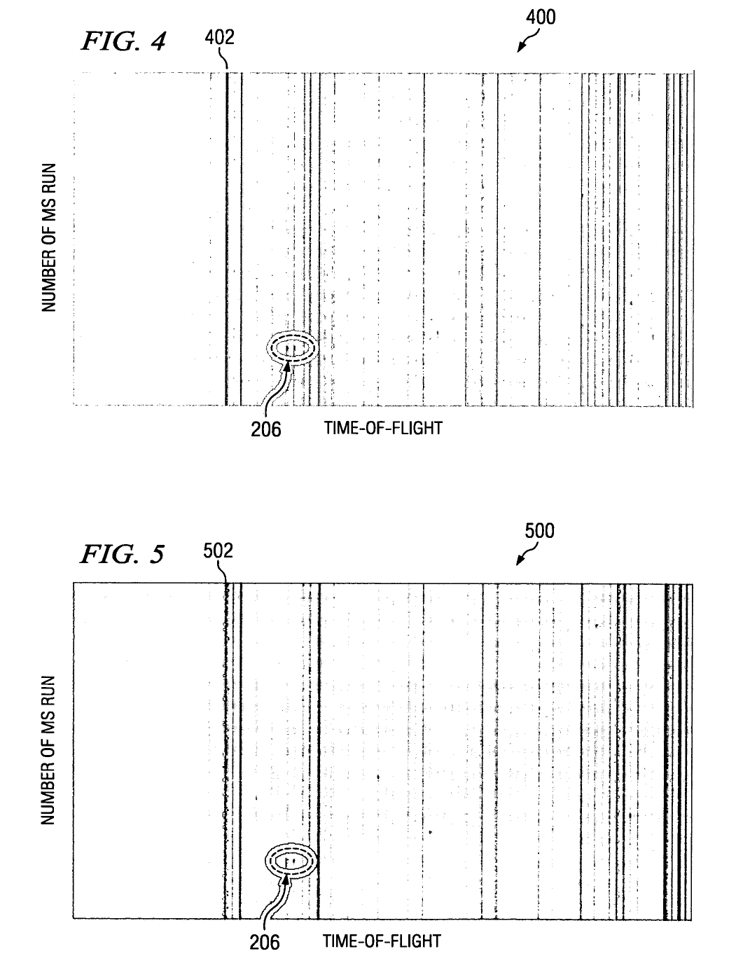

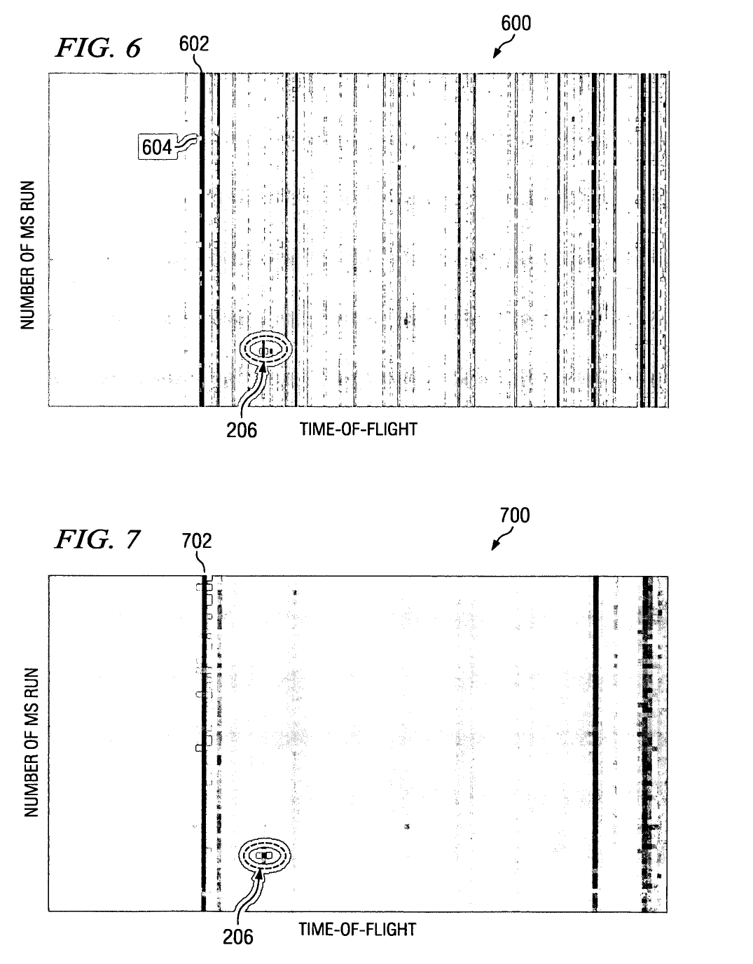

Referring now to FIG. 3, there is illustrated an exemplary time-of-flight mass spectrometer 300 that can be used in embodiments of the present invention. The mass spectrometer 300 includes a processing unit 302 operable to execute software 304. The processing unit 302 is in communication with a data acquisition unit 306 that is utilized to capture raw data produced by the time-of-flight mass spectrometer 300 as understood in the art. The processing unit 302 is further coupled to a memory 308 that may be utilized to receive and store raw data 307 and/or transformed data of the time-of-flight mass spectrometer 300. The memory 308 may be static, dynamic, electromagnetic, optical, or other storage media format. In certain embodiments, a display 310 may be coupled to the processor 302 and operable to receive and display the raw dataset 200 of FIG. 2 or transformed data (FIGS. 4-8). It should be understood that other types of data, such as the peak data signal 102 of FIG. 1, may also be displayed. In addition, it should be understood that the principles of the present invention may be applied to any type of mass spectrometer, and is not limited to the time-of-flight mass spectrometer described herein.

The software 304 may be operable to perform real-time processing of raw data 307 collected by the data acquisition unit 306. The software 304 utilizes lossless or lossy image processing techniques to reformat the raw data 307 collected by the data acquisition unit 306 into a hierarchical data format to provide for use at multiple resolutions without data loss. A hierarchical data format means that the data are transformed into a format that includes or stores increasingly higher resolutions in a nonredundant way. Such a storage format allows progressive retrieval with respect to resolution. Multiple resolution means that one has access to varying resolution levels of the data, in this case due to the storage format (i.e., in a hierarchical data format). In one embodiment, the image processing technique includes a wavelet transform as understood in the art. Additionally or alternatively, the wavelet transform may use a data-adaptive technique, which is an extension of conventional wavelet transforms and provides additional control of a variety of parameters for higher levels of data compression. The software 304 may also include compression and denoising algorithms that may be utilized to compress and/or denoise the transformed data in an unbiased and controlled manner. The multi-resolution representation allows for higher levels of data compression than if performed on the raw data 307 collected by the time-of-flight mass spectrometer 300 by utilizing custom-designed filters to represent irregular raw data 307 produced by the mass spectrometer. The hierarchical nature of the multi-resolution representation enables hierarchical data mining, storage, and retrieval functionality, for example. Further discussion of the software 304 may be found in conjunction with FIG. 11 hereinafter.

The hierarchical data format of the transformed data may be represented as a set of images that have increasingly higher coarsened levels (i.e., at multiple resolutions), as shown in FIGS. 4-7. Due to inherent properties of the wavelet transform embodied in the software 304, the transformed data at any resolution level may be analyzed using the same technologies and algorithms as may be applied to the raw data 307. However, because the transformed data may be selectively altered (e.g., reduced) in resolution, various applications, such as matching, may be performed significantly faster on the transformed data at a lower resolution than the full resolution of the raw data set 200 (FIG. 2) produced by the time-of-flight mass spectrometer 300. In the progression of FIGS. 4-7, small amplitude and small width features disappear first, while large amplitude features remain visible. The darker spectral lines 202 of FIG. 2 can be corresponded to spectral lines 402, 502, 602, and 702 of FIGS. 4-7, respectively. Additionally, the dark spot 206 is shown in each of the FIGS. 4-7, but as the resolution of each of FIGS. 4-7 is reduced, the dark spot 206 becomes more pronounced. The dark spot 206 of FIG. 2 is not immediately identifiable at full resolution, but the lower resolution image representations in FIGS. 4-7 make it easier to identify a chemical contamination or other aberration measured by the time-of-flight mass spectrometer 300.

In the different resolution images 400, 500, 600, and 700 of FIGS. 4-7, respectively, it may be seen that major spectral features (e.g., spectral lines 402, 502, 602, and 702) are preserved even on very coarse scales. In addition, because the major spectral features are maintained, hierarchical data mining applications, such as matching, may be effectively utilized. For example, it is feasible to utilize databases of protein mass spectra, convert them to the hierarchical format of the transformed data, and then classify them according to similarity on a coarse scale. Thereafter, all proteins with a given coarse level representation can be identified and reclassified on a finer scale. By increasing resolution for matching proteins or other compounds, continuing elimination of proteins that do not match any sample protein at increasing resolutions expedites such data mining efforts. The process may be reiterated until a unique classification of the sample protein is achieved. As understood in the art, the individual hierarchical matches may be qualified according to a “goodness-of-match” measure, as perfect matches are unlikely. Since the hierarchical data format of the transformed data provides for an intrinsic level of resolution, the goodness-of-match measure arises naturally.

Data Compression

Since the transformed data is formatted in a hierarchical data format, high compression ratios for lossless (bitwise reversible) compression of mass spectrometry data is possible. The hierarchical data format also allows for a simple, but useful, lossy compression scheme, if coarser resolution levels suffice for a particular application. Using wavelet transforms makes it possible to maintain different regions of the transformed data at distinct resolution levels. The user may predefine the region of interest, e.g., where the important features reside, and maintain those regions at higher resolutions than the rest of the transformed data. This multi-resolution ability allows for higher compression ratios than if the entire dataset were to be maintained at a single resolution.

In the lossless data compression case, a correlation structure of the transformed data may be utilized. To construct a compressed hierarchical representation of the raw data 307, a compression algorithm may follow the wavelet transform. The wavelet transform effectively decorrelates the levels on short image distances. TABLE 1 shows some typical data compression ratios utilizing the principles of the present invention. The data compression ratios are on average 60% higher than could otherwise be achieved utilizing a conventional data compression algorithm, such as WINZIP. One reason for such high data compression ratios is that the hierarchical data format of the transformed data is better suited for data compression than the data format of the raw data 307 collected by the time-of-flight mass spectrometer 300.

TABLE 1
Lossless Data Compression Comparisons Table

| | | | | | Hierar- |
|---|---|---|---|---|---|
| | | | | Conven- | chical |
| | | | Hierarchical | tional | Raw |
| | | Conventional | Data | Raw Data | Format |
| | | Raw Data | Format | Compres- | Compres- |
| | | Compression | Compression | sion | sion |
| File | Original Size | Size | Size | Ratio | Ratio |
| 1 | 199,479,464 | 50,729,905 | 30,230,000 | 3.93 | 6.60 |
| 2 | 40,223,225 | 10,690,932 | 6,482,090 | 3.76 | 6.21 |
| 3 | 17,466,972 | 4,955,400 | 2,961,493 | 3.52 | 5.90 |

Noise Identification and Reduction

In an ideal mass spectrometry setup, the data acquisition unit 306 delivers a pure mass spectrum convoluted with the instrument resolution function. In reality, there are many influences contaminating the resulting spectrum. One external source of noise arises from the sample itself. Chemical noise can give rise to spurious peaks and hinder the automatic detection of important compounds. The hierarchical format of the data makes it possible to analyze correlations between runs of the mass spectrometer, thereby enabling detection in marking of the noise. See, for example, the dark spot 206 on FIGS. 2 and 4-7. As long as there are only small traces of chemical noise present, the noise may be represented as localized peaks along the vertical axis, which is the mass spectrometer run number coordinate of FIG. 2. Given an external parameter describing the number of mass spectrometer runs needed for a peak to be real, the noise can be identified and the corresponding mass spectrometer run can be removed from the data.

Another noise source is system noise that arises from the mass spectrometer 300 itself. Such intrinsic system noise may be due to voltage fluctuations in the analog-to-digital (A/D) system, dead times of counter statistics, lost data packets in the data processing system, and other variables. As the amplitude of such noise is typically small, the detection of small amplitude peaks of a data signal becomes difficult for detection and the average value of the background increases considerably. For compression purposes, the noise has more drastic negative influences as it dramatically decreases correlation between pixels, (i.e., transformed data elements), thereby rendering the use of context dependent schemes very difficult. The hierarchical data format of the formatted data allows for decorrelation and makes it possible to include an optional noise removal process, if desired. Although the hierarchical data format retains the full information from the raw data 307 of the mass spectrometer 300 to allow for exact lossless reconstruction, noise removal is a lossy procedure. Therefore, if noise removal is utilized to reduce or eliminate noise collected by the mass spectrometer 300, the resulting data becomes lossy.

Due to the decorrelation property of the hierarchical format of the data, the mass distribution functions of the pixel values on the various scales become very closely Gaussian. This property allows for defining a set of standard deviations, σ, related to the half-width of these Gaussian distribution functions. A signal may be defined for those pixels that, given an externally chosen probability parameter, are incompatible in a statistical sense with the observed distribution functions. Since the intrinsic noise is most pronounced at small distance scales, a 1σ on a fine scale and a 0.5σ on a next coarser scale may be selected as cutoffs. Scales coarser than a 0.5σ may be left unmodified.

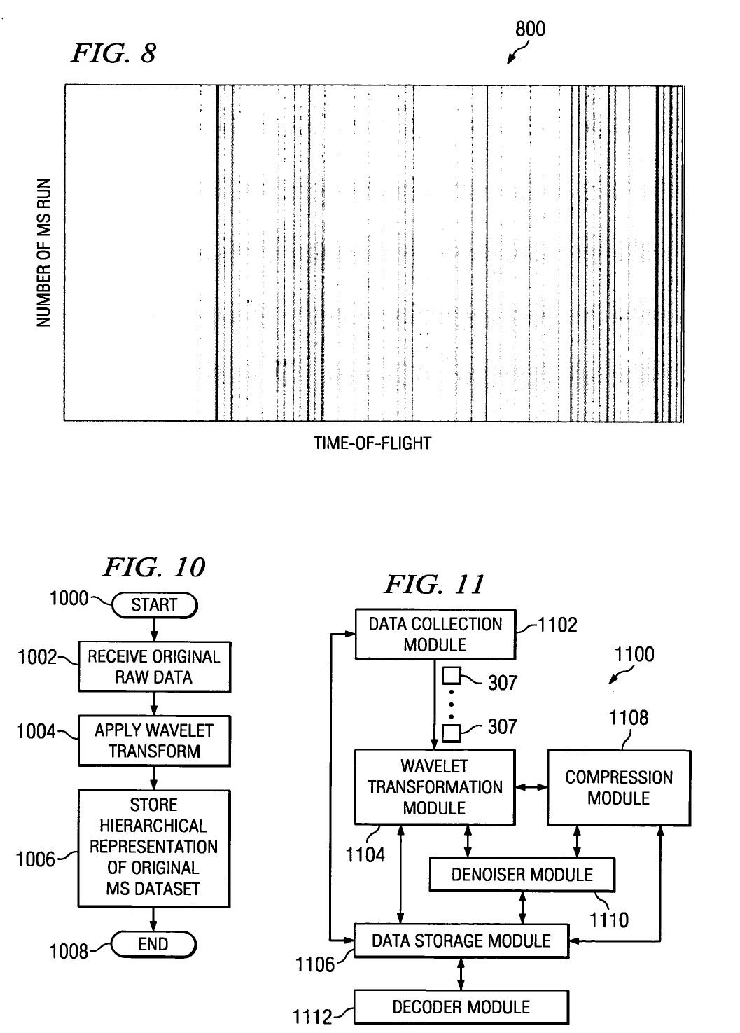

FIG. 8 is a graph of the raw dataset 200 of FIG. 2 having been denoised. As shown, the denoised image 800 resulting from denoising the raw dataset 200 as shown in FIG. 2 looks much clearer as the noise component of the signal is reduced and/or substantially removed. The spectral line 802, which corresponds to the spectral line 202, is thinner and clearer due to excess noise around the time-of-flight of the spectral line 802 being reduced or substantially eliminated.

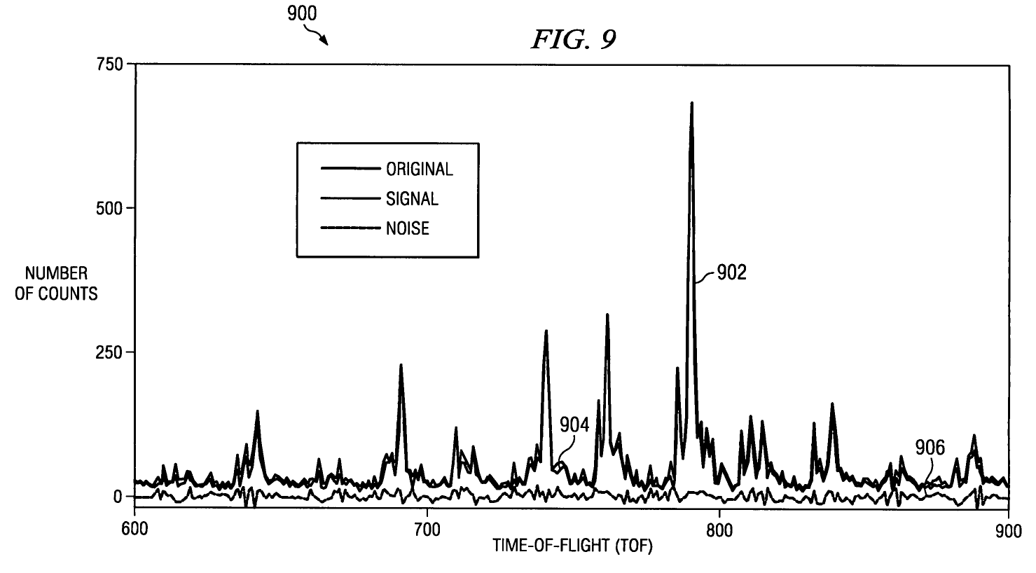

FIG. 9 is a graph 900 of exemplary peak data signal, including raw data, denoised data, and noise data, produced by the time-of-flight mass spectrometer 300 of FIG. 3. As shown, a raw peak data signal 902, which includes both signal and noise, denoised signal 904, and noise 906 are shown. At various points of the raw peak data signal 902, the noise 906 contributes fifty percent or more of the raw data signal 902, which makes it difficult to see low peaks in the signal 904 in some cases. As seen, the noise 906 is not purely additive, but multiplicative (i.e., the amplitude increases with the signal intensity). Such noise 906 makes it difficult to observe actual peaks in the raw peak data signal 902.

One problem with standard noise removal procedures is the removal of small features of the signal with the noise 906. This situation is problematic in the analysis of mass spectrometer data, where the dynamic range of the data may become very large. Because the principles of the present invention provide for formatting the data hierarchically, the wide dynamic range situations are handled with little or no loss of signal 904. The dynamical range of the raw data signal 902 over the time-of-flight range shown extends from small peaks having amplitudes of around ten counts to a large peak of over 650 counts. It has been shown that peaks as high as 2700 counts or more do not affect the dynamic range utilizing the principles of the present invention. As shown in FIG. 9, small peaks are visible even when the noise 906 is removed.

Algorithm Details

FIG. 10 is a flow diagram of an exemplary process for applying a wavelet transform to the raw data of mass spectrometer 300 of FIG. 3. The process starts at step 1000. At step 1002, raw data 307 measured by the time-of-flight mass spectrometer 300 is received. A wavelet transform is applied to the raw data at step 1004 to transform the raw data 307 into transformed data having the hierarchical data format.

In one embodiment, the wavelet transformation as applied at step 1004 utilizes nonseparable wavelets for two-dimensional datasets, such as those produced by a typical time-of-flight mass spectrometer 300. It should be noted that conventional wavelet transforms utilize separable wavelets in the case of transforming two-dimensional datasets. In the embodiment, the nonseparable wavelets may be defined using a dilation matrix D. The dilation matrix D may include two or more different dilation matrices, D₁ and D₂.

$${D}_{1} = ( \begin{matrix}2 & 0 \\ 0 & 1\end{matrix} ) , {D}_{2} = ( \begin{matrix}1 & 0 \\ 0 & 2\end{matrix} ) .$$

In the course of performing the wavelet transform, the two dilation matrices D₁ and D₂ are used either in a predefined intermittent order (e.g., use D₁ to obtain wavelet coefficients at coarsening level one, D₂ to obtain wavelet coefficients at coarsening level two, D₁ to obtain wavelet coefficients at coarsening level three, D₂ to obtain wavelet coefficients at coarsening level four, and so forth up to the highest coarsening level). Alternatively, an adaptive use of the dilation matrices may be utilized so that the choice of either dilation matrix D₁ or D₂ for each of the coarsening levels is made in the course of the wavelet transform depending on the properties of the raw data 307 being transformed. For n-dimensional datasets, the algorithm uses n dilation matrices D₁ . . . Dₙ with elements

(Dₖ)ᵢⱼ=δᵢⱼ(1+δₖᵢ).

For example, for three-dimensional datasets, the dilation matrices may be as follows:

$${D}_{1} = ( \begin{matrix}2 & 0 & 0 \\ 0 & 1 & 0 \\ 0 & 0 & 1\end{matrix} ) , {D}_{2} = ( \begin{matrix}1 & 0 & 0 \\ 0 & 2 & 0 \\ 0 & 0 & 1\end{matrix} ) , {D}_{3} = ( \begin{matrix}1 & 0 & 0 \\ 0 & 1 & 0 \\ 0 & 0 & 2\end{matrix} ) .$$

At step 1006, the transformed data having the hierarchical data format is stored. The process ends at step 1008.

FIG. 11 is a block diagram of exemplary software 304 for using a wavelet transformation to produce and store transformed data in a hierarchical data format from the raw data 307 collected by the mass spectrometer 300 of FIG. 3. As shown, the software 304 includes a data collection module 1102 that communicates the raw data 307 to a wavelet transformation module 1104. The wavelet transformation module 1104 may be in communication with a data storage module 1106, compression module 1108, and denoiser module 1110. Each of these modules 1106, 1108, and 1110 may further be in communication with each other as a user may elect to denoise, compress, and/or store the transformed data in a variety of ways. Further, a decoder module 1112 may be in communication with the data storage module 1106 to decode the transformed data at a selected resolution. It should be understood that the architecture of the software 304 may have alternative configurations and that the modules may alternatively be written as objects in an object-oriented software language, but perform substantially the same or functionally similar as a whole.

The wavelet transformation module 1104 is operable to perform a wavelet transformation in accordance with the principles of the present invention. The wavelet transformation module 1104 may utilize conventional wavelet transforms as well as a data-adaptive wavelet transform as discussed hereinbelow. Alternatively, the wavelet transformation module 1104 may be another type of image processing transformation that is operable to transform the raw data 307 into a hierarchical data format for use at multiple resolutions. The denoiser module 1110 may utilize any denoising algorithm as understood in the art. A simple denoiser may be utilized to disregard coefficients on the finer scales whose values are smaller than a predefined parameter. More sophisticated approaches may involve local estimation of a noise level using robust estimators, followed by soft or hard thresholding as described in the art. The compression module 1108 similarly may utilize any compression algorithm as understood in the art. In one embodiment, the compression algorithm may be a simple Huffman coder with context of varying sizes and variations thereof. It should be understood that the denoiser and compression algorithms are to be compatible with the hierarchical data format of the transformed data and that some denoiser and compression algorithms may be better suited and provide better results than others. Typically, however, such determination as to the quality of the denoising and compression is determined empirically as understood in the art. The data storage module 1106 is operable to store the data in the memory 308 of the time-of-flight mass spectrometer 300. Alternatively, the data storage module 1106 may store the data in a storage unit not part of the time-of-flight mass spectrometer 300. The decoder module 1112 may communicate with the data storage module 1106 to receive the transformed data, denoised data, and/or compressed data and decode the transformed data so as to enable a user to use the transformed data at a selected resolution.

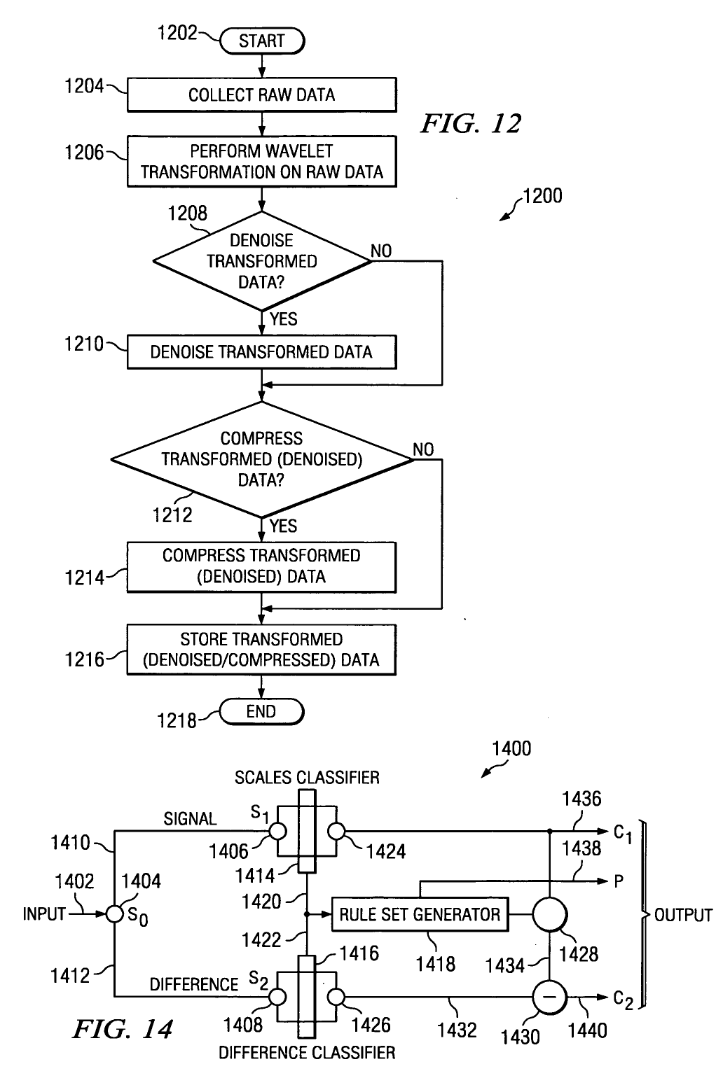

FIG. 12 is a flow diagram of an exemplary process for producing the transformed data having a hierarchical data format. The transformation process starts at step 1202. At step 1204, raw data 307 is collected by the time-of-flight mass spectrometer . At step 1206, an image processing algorithm is utilized to transform the raw data into transformed data in a hierarchical data format. In one embodiment, the image processing algorithm utilizes a wavelet transform. The wavelet transform may be a conventional wavelet transformation or a data-adaptive wavelet transform as discussed further below in connection with FIG. 14.

At step 1208, a determination is made as to whether to denoise the transformed data. If it is determined at step 1208 that the transformed data is to be denoised, then at step 1210, the transformed data is denoised. If it is determined at step 1208 that the transformed data is not to be denoised, then at step 1212, a determination as to whether the transformed (denoised) data is to be compressed is made. If it is determined that the transformed (denoised) data is to be compressed, then at step 1214, the transformed (denoised) data is compressed. At step 1216, the transformed (denoised/compressed) data is stored. If it is determined at step 1212 that the transformed (denoised) data is not to be compressed, then the process continues at step 1216 without compressing the transformed (denoised) data. The process ends at step 1218. After the data is stored, the transformed (denoised/compressed) data may be decoded by first decompressing, if compressed, and decoding for use at a desired resolution as discussed further herein.

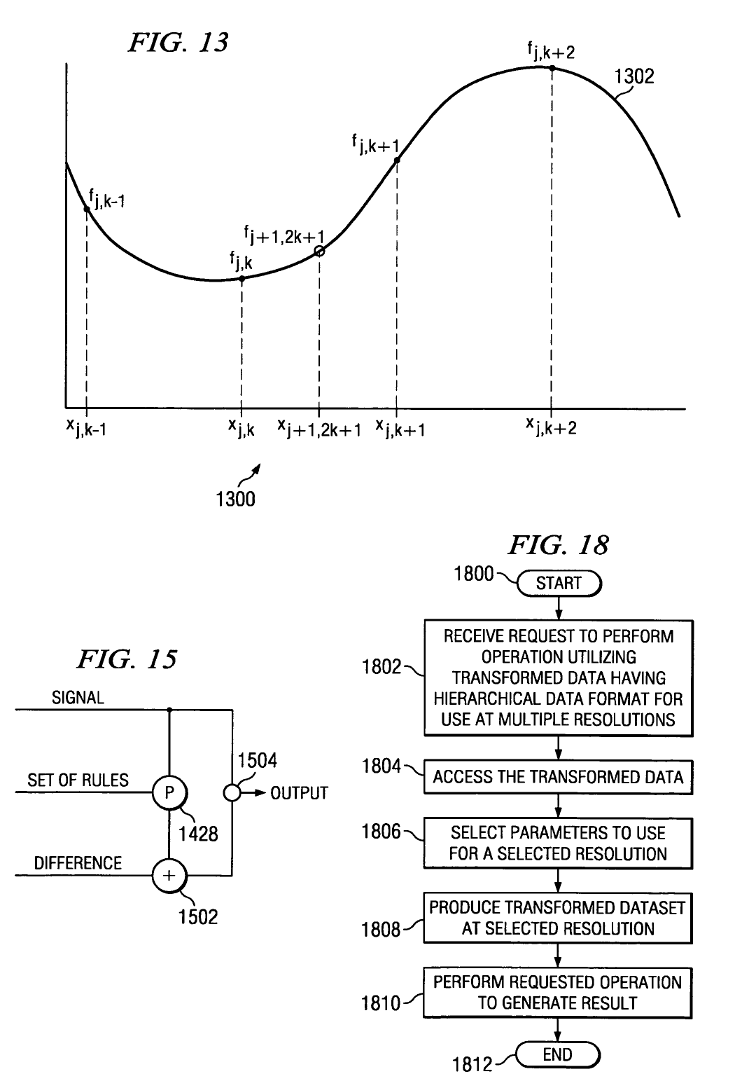

FIG. 13 is a graph showing an exemplary data signal for use in interpolating a data point on the data signal utilizing an interpolating polynomial. The solid circles are data points and the open circle is an interpolation point. An interpolating polynomial may be utilized to interpolate for the interpolation point. In one embodiment, the interpolating polynomial is a Lagrange interpolating polynomial as understood in the art. In establishing the interpolating polynomial, the following definitions and derivation are provided.

Compact support is defined as [−p+1, p−1].

φ is cardinal, i.e. φ(k)=δ_{0,k}, k ε Z. As a consequence, if the projection is defined onto Vⱼ via Pⱼf(x)=Σₖf_{j,k}φ_{j,k} (x), a one-to-one correspondence between (dyadic) grid points and basis functions results.

φ(x) is symmetric and is utilized for interpretation. A dilation equation is formed by construction as follows.

$$φ ( x ) = {\sum }_{- p + 1}^{p - 1} {g}_{k} φ ( 2 x - k ) = {\sum }_{- p + 1}^{p - 1} φ ( k / 2 ) φ ( 2 x - k )$$

and the gₖ, as defined below, are given in terms of the hₖ, the polynomial interpolation coefficients via

$${g}_{k} = \{ \begin{matrix}1 & k = 0 \\ 0 & k \neq 0 , k even \\ {h}_{( k + 1 ) / 2} & k \neq 0 , k odd \end{matrix}$$

If the original function values are taken from a polynomial of degree l<p, then the original function values may be reproduced (i.e. the interpolation may be represented by the polynomial P, again by construction).

$${∀}_{j , k} \{ \begin{matrix}{f}_{j + 1 , k } & = {f}_{j , k} \\ {f}_{j + 1 , 2 k + 1} & = {P}_{j + 1 , 2 k + 1}^{p} ( {x}_{j + 1 , 2 k + 1} )\end{matrix}$$

where P is a Lagrange interpolating polynomial of order p centered at (x_{j+1,2k+1}).

$${P}_{j + 1 , 2 k + 1}^{p} ( {x}_{j + 1 , 2 k = 1} ) = {\sum }_{l = k - p / 2 + 1}^{k + p / 2} {f}_{j , l} \frac{{\prod }_{{a = k - p / 2 + 1}_{a \neq 1}}^{k + p / 2} ( {x}_{j + 1 , 2 k + 1} - {x}_{j , s} )}{{\prod }_{{a = k - p / 2 + 1}_{a \neq l}}^{k + p / 2} ( {x}_{j , l} - {x}_{j , s} )}$$

in the case of x_{j,k}=Δ₀+kΔ(j), x_{j+1,k}−x_{j,k}=Δ(j)/2 (and substituting l=l−k ), the following function ƒ is obtained,

$${f}_{j + 1 , 2 k + 1} = {\sum }_{l = - p / 2 + 1}^{p / 2} {h}_{l} {f}_{j , k + 1} = {\sum }_{l = 1}^{p / 2} {h}_{l} ( {f}_{j , k - l + 1} + {f}_{j , k + l} )$$

where the last equality derives from symmetry, and

$${h}_{l} = {( - 1 )}^{p / 2 + l - 1} \frac{{\prod }_{i = 0}^{p - 1} ( i - p / 2 + 1 / 2 )}{( l - 1 / 2 ) ( p / 2 + l - 1 ) ! ( p / 2 - l ) !} - p / 2 < l \leq p / 2$$

These coefficients can be calculated for any interpolation order and can then be reused in the actual transform.

A fast lifted interpolating wavelet transform as understood in the art may be utilized in providing for the principles of the present invention. The fast lifted interpolating wavelet transform may be provided in d dimensions. For simplicity, a d-dimensional analog of the row-column transform defining the dilation matrices may be utilized, where the dilation matrix D is described as,

$${D}_{i} = ( \begin{matrix}⋰ & & & & & & \\ & 1 & & & & 0 & \\ & & ⋰ & & & & \\ & & & 2 & & & \\ & & & & ⋰ & & \\ & 0 & & & & 1 & \\ & & & & & & ⋰\end{matrix} ) ,$$

which are unit matrices with a value of 2 on the i^{th} position along the diagonal, and the corresponding digit vectors e₁=( . . . ,0, . . . ,1, . . . ,0, . . . ), which are zero with a value of 1 on position i. The transforms are parameterized by the sequence of dilations Dᵣ_{₁}Dᵣ_{₂} . . . Dᵣ_{_{L}} and hence by the L-tuple (r₁,r₂ . . . , r_{L}). Since a different filter may be used for each subdivision, this tuple L, together with a corresponding tuple of filters, specifies the transform. These parameters may be set in the input to the algorithm. The fast lifted interpolating wavelet transform may then be written as,

$${s}_{kj}^{j} = {s}_{{D}_{{r}_{j}} {k}_{j}}^{j + 1}$$

$${d}_{kj}^{j} = {s}_{{D}_{{r}_{j}} {k}_{j} + {e}_{{r}_{j}}}^{j + 1} - {\sum }_{1} {h}_{l} {s}_{{D}_{{r}_{j}} {k}_{j} + 2 {le}_{{r}_{j}}}^{j + 1}$$

again making use of the above derived coefficients.

Data-Adaptive Wavelets

In another embodiment, a data-adaptive wavelet transform may be utilized in accordance with embodiments of the present invention. A data-adaptive wavelet provides an algorithm that attempts to optimize the filters given the local, coarse-grained environment. The optimization is over a suitable choice of classifiers. As an example, the position of an interpolating polynomial with respect to the location of the interpolation may be altered. For example, if four points are used for the interpolation, two points may be selected on the left side of the point of interpolation and two on the right of the point of interpolation. Alternatively, three points can be positioned on one side of the point of interpolation and one point can be positioned on the other side. Depending on the selection of the classifiers, the optimization of the filters may be improved to provide for better interpolations, thereby improving the structure of the data after the transformation with respect to compressibility and denoising. The optimization criteria used below is chosen such as to render coefficients in the transformed data as small as possible leading to smaller symbolsets and therefore to better compression. In determining the classification space, the location of the interpolating polynomial with respect to the coordinate of the interpolated point may be defined. For the polynomial P below is solved in 1-dimension for a scanline-by-scanline pass and may easily be generalized to higher dimensions using the deBoer-Ron algorithm as understood in the art.

More specifically, an interpolating polynomial of order p evaluated at position l (i.e. P^{p.l}) may be chosen to restrict the possible shifts to lie symmetrically around the center and to include the ordinate of the point to be interpolated. For example, for p=2 (linear interpolation), there is a shift to the left, the center, and a shift to the right [symbol] l=1,2,3

$${P}^{2 , 1} = \frac{3}{2} {f}_{1} - \frac{1}{2} {f}_{3} \qquad {P}^{2 , 2} = \frac{1}{2} {f}_{- 1} + \frac{1}{2} {f}_{1} \qquad and \qquad {P}^{2 , 3} = - \frac{1}{2} {f}_{- 3} + \frac{3}{2} {f}_{- 1}$$

where the ƒ* are the function values at position * relative to the interpolatee.

FIG. 14 illustrates the production of the transformed data having a hierarchical data format utilizing a data-adaptive wavelet transform as may be performed by the software 304 of FIG. 11. The block diagram includes an input line 1402 coupled to node 1404. The node 1404 is coupled to two different nodes 1406 and 1408 via lines 1410 and 1412, respectively. Node 1406 is an input to a scales classifier block 1414 for finding a vector of optimal classification indices on scales. Node 1408 is an input to a difference classifier block 1416 for finding a vector of optimal classification indices on differences. The classifier blocks 1414 and 1416 have outputs that are coupled to a rule set generator 1418 via lines 1420 and 1422, respectively. Each of the classifier blocks 1414 and 1416 have output nodes 1424 and 1426, respectively. The rule set generator 1418 has an output that is coupled to a predictor (P) or polynomial block 1428. A subtractor 1430 receives inputs from the outputs of the difference classifier block 1416 and the predictor block 1428 via lines 1432 and 1434, respectively. The outputs of the data-adaptive wavelet transform include the outputs of the scales classifier block 1414, rule set generator 1418, and subtractor 1430 via lines 1436, 1438, and 1440, respectively.

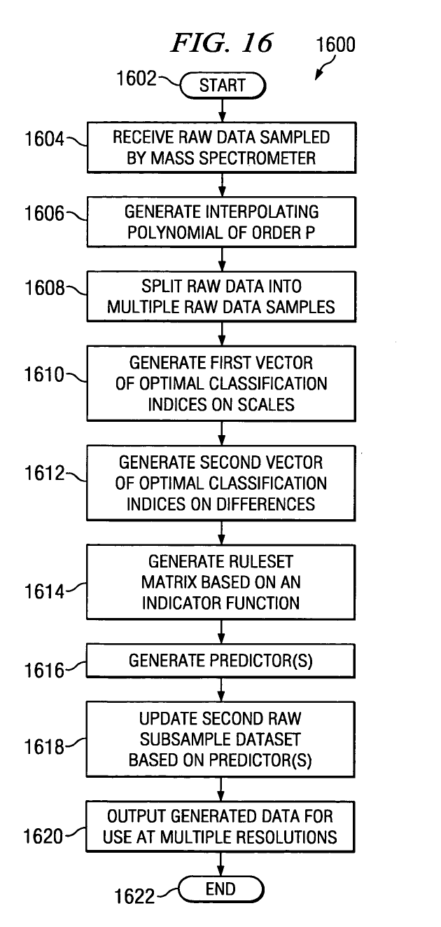

Referring now to FIG. 16, a flow chart generally describing an exemplary method for generating the transformed data having a hierarchical data format by utilizing a data-adaptive wavelet transform as illustrated by the block diagram of FIG. 14 is shown. The process starts at step 1602. At step 1604, the raw data 307 sampled by the mass spectrometer 300 is received at node 1404. An interpolating polynomial of order p is generated at step 1606. At step 1608, the raw data 307 received at the node 1404 is split into multiple raw data samples or subsamples, a signal subsample being applied to the scales classifier block 1414 and a difference subsample being applied to the difference classified block 1416. In one embodiment, the raw data may be split into even and odd samples and stored in separate arrays.

At step 1610, a first vector of optimal classification indices on scales is generated. A second vector of optimal classification indices on differences is generated at step 1612. At step 1614, a ruleset matrix based on an indicator function is generated. In one embodiment, the indicator function is a MAXARG function. Predictor(s) are generated at step 1616, where the predictor(s) are utilized to update the second vector or difference subsample dataset at step 1618. At step 1620, the generated data, including the first vector, updated second vector, and ruleset matrix, for use at multiple resolutions is output at step 1620. The process ends at step 1622. The data that is output may thereafter be decoded and utilized at a selected resolution.

In summary, and at a very high level, the method for generating the transformed data may be performed by the following process elements, which are described in detail with regard to the continuing description of FIG. 14 below.

1. Split input signal

2. Classification on scales

3. Classification on differences

4. Generation of ruleset

5. Prediction

6. Output

1. Split Input Signal

Referring again to FIG. 14, in detailed operation, the input line 1402 receives an input signal S₀, which enters node 1404. The input signal S₀ is defined as S₀={s₁, . . . ,S_{N}} of length N, order p, and classification space l=1, . . . ,p+1. The node 1404 splits the input signal S₀ into two subsamples, S₁ and S₂, where subsample S₁ is formed from the odd samples of the input signal S₀ (i.e., S₁={s₁,s₃, . . . }:={s₁¹, s_{N/2}¹}) and subsample S₂ is formed from the even samples of the input signal S₀ (i.e., S₂=d₁={s₂,s₄, . . . }:={d₁¹, . . . d_{N/2}¹}). This splitting make use of the special structure of the dilation matrices defined above, such that only one dimensional operations are involved. However, the transform as a whole may be extended for multidimensional operations.

2. Classification on Scales

The scales classifier block 1410 is operable to find a vector of optimal (over l) classification indices on scales by performing the following:

j^{s}={j₁^{s}, . . . ,j_{N/2}⁸}

jᵢ^{s}=argmin [ƒ(l)=|sᵢ¹−P^{p,l}(in s₁)|]

3. Classification on Differences

The scales classifier block 1412 is operable to find a vector of optimal (over l) classification indices on differences by performing the following:

j^{d}={j₁^{d}, . . . ,j_{N/2}^{d}}

j₁^{d}=argmin [ƒ(l)=|dᵢ¹−P^{p.l}(in dₗ)|]

4. Generation of Ruleset

An indicator function is defined as I^{m,l}(k), k=1, . . . ,p+1, which gives the number of times in d₁ the predictor of index k is optimal given that its neighbors in S₁ have optimal predictors m and l, i.e.,

$${I}^{m , l} ( k ) = {\sum }_{i = 1}^{N / 2} {δ}_{{j}_{i}^{d} , k} {δ}_{{j}_{i}^{s} , m} {δ}_{{j}_{i + 1}^{s} , l}$$

For each neighborhood (m,l) find the k that maximizes I^{m,l}(k); the resulting rule matrix gives the locally optimal rule set for prediction on d's if only s's (and the rule matrix) are available as prior knowledge:

P_{m,l}=argmax I^{m,l}(k)

5. Prediction

Given the index vector on scales j^{s}, and a position in dₗ, e.g. dᵢ^{l}, the neighbor classifiers (m*,l*) are found to obtain a likely estimate for an optimal predictor for dᵢ¹ via k*=P_{m*,l*.} P^{p,k*} is formed to perform the update on the difference signal, S₂ (dᵢ^{l}).

6. Output

The ruleset p, which is a (p+1)×(p+1) matrix, the signal s¹ (i.e., C₁) and the updated d¹ (i.e., C₂). These outputs provide for the hierarchical data format produced by the data-adaptive wavelet transform.

Referring now to FIG. 15, a representative diagram illustrating a decoder 1500 utilized to receive the output of FIG. 14 to reproduce a dataset transformed by the data-adaptive wavelet transform is provided. The decoder 1500 utilizes the predictor (P) block 1428, which is coupled to a summer 1502. The predictor block 1428 receives the signal s¹ and rulesetp. The output of the predictor block 1428 is input into the summer 1502, which adds the output to the updated difference d¹. An output node 1504 is utilized to produce the transformed data having the resolution as selected. Inputs to the output node 1504 include the signal s¹ and output of the summer 1502. This process for selecting a resolution may be iterated starting from the coarsest scale and differences, generating the next coarser scale, using the transmitted (stored) difference to generate the next scale, and so forth, until the original transformed data is recovered. The directions are defined by the sequence of dilation matrices with which the original transformed data were transformed.

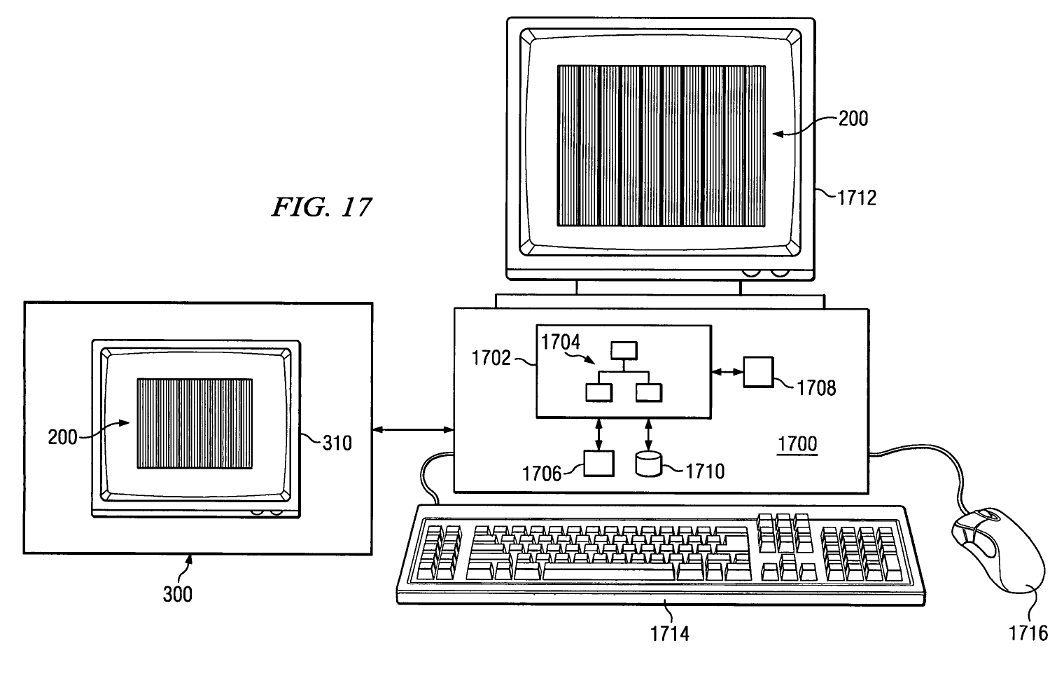

FIG. 17 is a block diagram of a time-of-flight mass spectrometer 300 in communication with a computing system 1700, where the computing system 1700 is utilized to receive and use the transformed data for one or more operations as desired by a researcher, for example, utilizing the time-of-flight mass spectrometer 300. The computing system 1700 includes a processor 1702 operable to execute software 1704. The processor 1702 may be coupled to a memory 1706 for storage of the transformed data. The processor 1704 may further be coupled to an input/output (I/O) unit 1708 and a storage unit 1710, such as a disk drive, where the disk drive is operable to store the transformed data 307 while not being utilized.

The computing system 1700 may further include a display 1712 for displaying the raw or transformed data 200 so as to enable a researcher to view the transformed data at a selected resolution. The computing system 1700 may further include control devices, such as a keyboard 1714 and a mouse 1716. The control devices 1714 and 1716 may be utilized to control uses of the transformed data, such as selecting a resolution to view the transformed data. Alternatively, control devices incorporated into the time-of-flight mass spectrometer 300 may be utilized to control selection of the resolution of the transformed data.

FIG. 18 is a flow diagram of an exemplary procedure for using the transformed data in the hierarchical data format collected by the mass spectrometer of FIG. 17 for a variety of operations. The process for utilizing the transformed data starts at step 1800. At step 1802, a request to perform an operation utilizing the transformed data having a hierarchical data format for use at multiple resolutions is received. The request may be initiated by a user of the computing system 1700 or automatically initiated as the transformed data is received by the computing system 1700. In an alternative embodiment, the time-of-flight mass spectrometer 300 communicates raw data 307 to the computing system 1700 rather than the transformed data and the computing system 1700 performs the transformation of the raw data 307 into transformed data having a hierarchical format.

At step 1804, the transformed data is accessed. In one embodiment, the transformed data 307 may be accessed on the computing system 1700 in either the memory unit 1704 or storage unit 1710 for access directly from the time-of-flight mass spectrometer 300. At step 1806, parameters to use for a selected resolution may be selected by a user of the computing system 1700 or time-of-flight mass spectrometer 300. In one embodiment, the user of the computer system 1700 may select the resolution parameters by typing while using the software 1704. Alternatively, the user may select the resolution parameters via a graphical user interface as understood in the art.

At step 1808, using the decoder module 112 with the selected resolution parameters produces the transformed data at the selected resolution. The available resolutions are defined by the rescaling through the dilation matrices, and as such involve powers of two (provided by the dilation matrices) in the various directions. Finer gridding of the available resolution levels may be obtained by using a multiwavelet transform as described in the art. At step 1810, the requested operation is performed to generate a result. The requested operation may include searching, matching, displaying, or other function desired by the user to assist in performing one or more research operations on the data collected by the time-of-flight mass spectrometer 300. The process ends at step 1812.

As will be recognized by those skilled in the art, the innovative concepts described in the present application can be modified and varied over a wide rage of applications. Accordingly, the scope of patents subject matter should not be limited to any of the specific exemplary teachings discussed, but is instead defined by the following claims.

We claim:

1. A mass spectrometer system, comprising:
a data acquisition unit operable to sense and generate raw data indicative of masses of particles; and
a computing unit in communication with said data acquisition unit and configured to receive the raw data from said data acquisition unit and transform the raw data into transformed data having a hierarchical data format for use at multiple resolutions.

2. The mass spectrometer system according to claim 1, wherein said computing unit is further configured to compress the transformed data.

3. The mass spectrometer system according to claim 2, wherein said computing unit uses a lossless compression technique to compress the transformed data.

4. The mass spectrometer system according to claim 1, wherein said computing unit is further configured to identify noise in the transformed data.

5. The mass spectrometer system according to claim 4, wherein said computing unit is further configured to reduce the noise in the transformed data.

6. The mass spectrometer system according to claim 1, further comprising a display unit in communication with said computing unit and operable to display the transformed data at multiple resolutions.

7. The mass spectrometer system according to claim 1, wherein said computing unit utilizes a wavelet transformation having filters that transform the raw data into the transformed data.

8. The mass spectrometer system according to claim 7, wherein said processing unit is further operable to optimize the filters used in the wavelet transformation.

9. The mass spectrometer system according to claim 8, wherein said processing unit is further operable to generate multiple sub-datasets from the raw data.

10. The mass spectrometer system according to claim 9, wherein the multiple datasets include a first dataset formed of odd indexed elements of the raw data and a second dataset formed of even indexed elements of the raw data.

11. The mass spectrometer system according to claim 9, wherein said processing unit is configured to determine classifiers for optimizing the filters.

12. The mass spectrometer system according to claim 11, wherein the classifiers include classifiers for scales and differences.

13. The mass spectrometer system according to claim 11, wherein said processing unit is further configured to generate a rule set for optimizing the filters.

14. The mass spectrometer system according to claim 13, wherein the rule set includes a function for taking a maximum of an argument.

15. The mass spectrometer system according to claim 13, wherein said processing unit is further configured to obtain an estimate for an optimal predictor based on the classifiers to produce an interpolation point from the transformed data.

16. The mass spectrometer system according to claim 1, wherein said processing unit further includes a decoder to decode the transformed data utilizing the rule set.

17. A method for storing mass spectrometer data, said method comprising:
receiving raw data indicative of masses of particles produced by a mass spectrometer; and
transforming the raw data into transformed data having a hierarchical data format for use at multiple resolutions.

18. The method according to claim 17, further comprising compressing the transformed data.

19. The method according to claim 18, wherein said compressing the transformed data is performed using a lossless compression technique.

20. The method according to claim 17, further comprising identifying noise in the transformed data.

21. The method according to claim 20, further comprising reducing the noise in the transformed data.

22. The method according to claim 21, further comprising compressing the transformed data having reduced noise.

23. The method according to claim 17, further comprising displaying the transformed data at multiple resolutions.

24. The method according to claim 17, wherein said transforming includes performing a wavelet transformation on the raw data to produce the transformed data having a hierarchical data format.

25. The method according to claim 17, further comprising decoding the transformed data.

26. A mass spectrometer system, comprising:
means for receiving raw data indicative of masses of particles; and
means for transforming the raw data into transformed data having a hierarchical data format for use at multiple resolutions, said means for transforming being in communication with said means for generating.

27. The method according to claim 26, further comprising means for compressing the transformed data.

28. The method according to claim 26, further comprising means for identifying noise in the transformed data.

29. The method according to claim 28, further comprising means for reducing the identified noise in the transformed data.

30. The method according to claim 29, further comprising means for compressing the transformed data having reduced noise.

31. The method according to claim 26, further comprising means for displaying the transformed data at multiple resolutions.

32. The method according to claim 26, further comprising means for decoding the transformed data.

33. A method for processing mass spectrometry data, said method comprising:
receiving a request to perform an operation utilizing at least a portion of transformed data resulting from a transformation of raw data generated by a mass spectrometer, the transformed data having a hierarchical data format for use at multiple resolutions;
accessing the transformed data;
selecting parameters to use for a selected resolution of the transformed data;
producing a transformed dataset at the selected resolution from the transformed data as a function of the selected parameters; and
performing the requested operation on the transformed dataset at the selected resolution to generate a result for the operation based on the transformed dataset at the selected resolution in response to said receiving the request.

34. The method according to claim 33, wherein said receiving the request includes receiving a request to compare a test dataset with the transformed data.

35. The method according to claim 33, wherein said receiving the request includes receiving a search request for transformed data having certain properties.

36. The method according to claim 33, wherein said receiving the request includes receiving a request to compress the transformed data.

37. The method according to claim 33, wherein said receiving the request includes receiving a request to identify noise contained in the transformed data.

38. The method according to claim 37, wherein said receiving the request includes receiving a request to identify chemical noise contained in the transformed data.

39. The method according to claim 37, further comprising receiving a request to suppress the noise.

40. The method according to claim 33, further comprising decoding the transformed data at the selected resolution.

41. A system for processing mass spectrometry data, said system comprising:
a storage unit operable to store transformed data resulting from a transformation of raw data generated by a mass spectrometer, the transformed data having a hierarchical data format for use at multiple resolutions; and
a processing unit in communication with said storage unit and configured to:
receive a request to perform an operation utilizing at least a portion of transformed data resulting from a transformation of raw data generated by a mass spectrometer, the transformed data having a hierarchical data format for use at multiple resolutions;
access the transformed data;
select parameters to use for a selected resolution of the transformed data;
produce a transformed dataset at the selected resolution from the transformed data as a function of the selected parameters; and
perform the requested operation on the transformed dataset at the selected resolution to generate a result for the operation based on the transformed dataset at the selected resolution in response to receiving the request.

42. The system according to claim 41, wherein the operation includes comparing a test dataset with the transformed data.

43. The system according to claim 41, wherein the operation includes searching for transformed data having certain properties.

44. The system according to claim 41, wherein said processing unit is further operable to compress the transformed data.

45. The system according to claim 41, wherein the operation includes identifying noise contained in the transformed data.

46. The system according to claim 45, wherein the noise is chemical noise.

47. The system according to claim 45, wherein the operation includes suppressing the noise.

48. The system according to claim 41, wherein said processing unit is further operable to decode the data at the selected resolution.

49. A method for formatting data measured by a mass spectrometer, said method comprising:
receiving raw data sampled by the mass spectrometer;
generating an interpolating polynomial of order p for use in generating coefficients;
splitting the raw data into multiple raw subsample datasets;
generating a first vector of optimal classification indices on scales;
generating a second vector of optimal classification indices on differences;
generating a ruleset matrix based on an indicator function;
generating a predictor as a function of the ruleset, first vector, and second vector;
based on each predictor, updating the second raw subsample dataset utilizing the coefficients; and
outputting the ruleset matrix, first raw subsample dataset, and updated second raw subsample dataset for use of the data measured by the mass spectrometer at multiple resolutions.

50. The method according to claim 49, wherein said splitting of the raw data includes forming two raw subsample datasets, a first dataset including odd indexed raw data elements and a second dataset including even indexed raw data elements.

51. The method according to claim 49, wherein said generating the ruleset matrix is performed by utilizing a maximum of an argument (MAXARG) function.

52. The method according to claim 49, further comprising compressing the datasets.

53. The method according to claim 49, further comprising:
identifying noise included in the raw data; and
suppressing the identified noise.

54. A system for formatting data measured by a mass spectrometer, said system comprising:
means for receiving raw data sampled by the mass spectrometer;
means for generating an interpolating polynomial of order p for use in generating coefficients and in communication with said means for receiving;
means for splitting the raw data into multiple raw subsample datasets, and in communication with said means for receiving;
means for generating a first vector of optimal classification indices on scales, and in communication with said means for splitting to receive the multiples raw subsample datasets to generate the first vector;
means for generating a second vector of optimal classification indices on differences, and in communication with said means for splitting to receive the multiple raw subsample datasets to generate the second vector;
means for generating a ruleset matrix based on an indicator function, and in communication with said means for splitting to receive the multiple raw subsample datasets to generate the ruleset matrix;
means for generating a predictor as a function of the ruleset matrix, first vector, and second vector, operable to receive the ruleset matrix first vector, and second vector;
means for updating the second raw subsample dataset utilizing the coefficients and in response to each predictor; and
means for outputting the ruleset matrix, first raw subsample dataset, and updated second raw subsample dataset for use of the data measured by the mass spectrometer at multiple resolutions.

55. The system according to claim 54, further comprising means for compressing the datasets in communication with said means for outputting.

56. The system according to claim 54, further comprising:
means for identifying noise included in the raw data and operable to receive the raw data or the multiple raw subsample datasets; and
means for suppressing the identified noise in communication with said means for identifying noise.

57. A method for formatting data measured by a mass spectrometer, said method comprising:
receiving a dataset containing mass spectrometer data;
performing a wavelet transformation on the mass spectrometer data to generate a transformed dataset; and
storing the transformed dataset.

58. The method according to claim 57, further comprising compressing the transformed dataset.

59. The method according to claim 57, further comprising suppressing noise contained in the transformed dataset.

60. The method according to claim 57, further comprising suppressing noise contained in the transformed dataset.

61. The method according to claim 57, further comprising optimizing filters over localized regions.

62. The method according to claim 61, further comprising generating a ruleset for performing predictions in interpolating datapoints.

63. A system for formatting data measured by a mass spectrometer, said system comprising:
a processor operable (i) to receive a dataset containing mass spectrometer data and (ii) to perform a wavelet transformation on the mass spectrometer data to generate a transformed dataset; and
a storage unit in communication with said processor and operable to receive and store the transformed dataset communicated from said processor.

64. The system according to claim 63, wherein said processor is further operable to compress the transformed dataset.

65. The system according to claim 64, wherein said processor is further operable to suppress the noise contained in the transformed dataset.

66. The system according to claim 63, wherein said processor is further operable to suppress the noise contained in the transformed dataset.

67. The method according to claim 63, further comprising optimizing filters over localized regions.

68. The method according to claim 67, further comprising generating a ruleset for performing predictions for interpolating datapoints.
